(19) United States
(12) Patent Application Publication
(10) Pub. No.: US 2007/0073210 A1
(43) Pub. Date: Mar. 29, 2007

(54) Wound dressing and adhesive wound dressing comprising a vasoconstrictive ingredient, and processes for the production thereof

(75) Inventors: Thomas Hille, Neuwied (DE); Christian Hoffmann, Neuwied (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 10/584,477

(22) PCT Filed: Dec. 9, 2004
(86) PCT No.: PCT/EP04/13996

§ 371 Date: Jun. 23, 2006

(30) Foreign Application Priority Data
Dec. 24, 2003 (DE) 103 61 306.4

(51) Int. Cl. A61F 13/00

(52) U.S. Cl. 602/48; 602/43

(57) ABSTRACT

A wound dressing provided as a ready-made product for covering bleeding wounds. The wound dressing comprises a carrier material containing at least one vasoconstrictive medicinal substance.

### CROSS-REFERENCE TO RELATED APPLICATIONS

This application is a National Stage application of International Application No. PCT/EP2004/013996, filed on Dec. 9, 2004, which claims priority of German application number 103 61 306.4, filed on Dec. 24, 2003.

### BACKGROUND OF THE INVENTION

1. Field of the Invention

The present invention concerns wound dressings and adhesive wound dressings for covering and treating bleeding wounds. It furthermore relates to processes for producing wound dressings and adhesive wound dressings.

2. Description of the Prior Art

To treat bleeding wounds, for example cuts, lacerations or scratches in the skin, one usually utilises wound dressings or wound plasters (also called adhesive wound dressings), which cover the wound towards the outside and absorb the issuing blood. Not infrequently, the bleeding ceases only after an extended period of time, so that repeated changing of the dressing is required. For other reasons, too, it may be desirable to staunch the bleeding as soon as possible.

From the field of boxing it is known to use adrenaline for treating bleeding wounds, the treatment being carried out in such a manner that an adrenaline solution is applied to the wound to be treated by an applicator made of absorbent material (e.g. cotton swabs) which has previously been impregnated with the adrenaline solution. As a result of the vasoconstrictive action of the adrenaline, the bleeding is thereby, as a rule, staunched directly and quickly.

This type of wound management is, however, not suitable for daily use since adrenaline—as well as the salts thereof—is very instable and quickly disintegrates upon exposure to oxygen (e.g. aerial oxygen) and light. The decomposition product formed by this reaction is called adrenochrome; it is of an intense red colour and can trigger hallucinations and schizophrenia-like states. Because of these properties, even minor proportions of this decomposition product affect the quality of an adrenaline-containing pharmaceutical product to such an extent that the latter does not meet the criteria for approval of pharmaceuticals. Due to the mentioned intense red colour, even traces of decomposed adrenaline can be detected easily. Because of this known instability of adrenaline, it is used in the form of stabilised solutions which are directly applied to the wounds in liquid form—as in the above-described example.

### SUMMARY OF THE INVENTION

It was thus the object of the present invention to provide wound dressings and adhesive wound dressings which possess the advantages of the rapid blood staunching that can be achieved with adrenaline, whereas the disadvantages caused by the instability of adrenaline are to be avoided. More particularly, the object was to make the medicinal substance adrenaline available for wound treatment in a form other than the liquid form, and to facilitate its use for this treatment purpose. A further object of the invention was to indicate processes for producing wound dressings and adhesive wound dressings of the afore-mentioned type.

Surprisingly, this object is achieved by wound dressings and adhesive wound dressings according to the present invention.

According to the present invention, a wound dressing for covering bleeding wounds comprises a carrier material (also called a reservoir) containing at least one vasoconstrictive medicinal substance. These wound dressings are provided as ready-made products and can be produced by large-scale production. Thereby, a wound dressing material is provided which affects the rapid staunching of the bleeding and which is easy to handle. The wound dressing, which is impregnated with a vasoconstrictive medicinal substance, is taken out of its package and is directly applied to the wound to be treated. If required, it can be fixated there by suitable means (e.g. bandage, plaster). In many cases, the bleeding stops already after a few minutes, so that a long-lasting fixation is not necessary.

The wound dressings or adhesive wound dressings according to the invention may either contain a single medicinal substance with vasoconstrictive action, or a combination of at least two such medicinal substances. Suitable as vasoconstrictive medicinal substances are, in particular, active substances from the group of the sympathomimetics, for example adrenaline and noradrenaline, adrenaline being most preferred. The medicinal substances can also be used in the form of their respective pharmaceutically acceptable salts or addition compounds, with adrenaline HCl and adrenaline acid tartrate, as well as noradrenaline HCL and noradrenaline acid tartrate being most preferred.

The medicinal substance, or the combination of medicinal substances, is generally present in the wound dressing in solid form, i.e. the carrier material is impregnated with the medicinal substance or the combination of medicinal substances and the medicinal substance(s) is/are adsorbed to the carrier material. The content of medicinal substance is preferably 0.01 to 25%-wt., particularly 0.1 to 10%-wt., especially preferably 0.1 to 5%-wt., in each case relative to the carrier material wherein the medicinal substance(s) is/are contained.

According to another embodiment of the invention, the wound dressing or the adhesive wound dressing, preferably the carrier material thereof, contains in addition at least one astringent or/and haemostatic substance. Suitable substances for this purpose are, in particular, those of the following group: tannins, aluminium salts, zinc salts, calcium salts; Al-hydroxychlorides, K-Al-sulphate, ammonium-Al-sulphate; iron compounds, gelatine, collagen, thromboplastin, thrombin.

It may, furthermore, be advantageous to add at least one additional active substance which promotes the healing of the wound but has no vasoconstrictive effect. Suitable for this purpose are, above all, amino acids, especially glycine, as well as peptides, enzymes, lymphokines, coagulation factors, anti-inflammatory substances (e.g. bisabolol, camomile extracts), vitamins (especially vitamin A, vitamin B1, vitamin B3, vitamin B5, vitamin B6, vitamin E), polysaccharides and skin caring substances (e.g. dexpanthenol, panthenol, pantothenic acid, allantoin, aloe Vera and other plant extracts; protein hydrolysates, albumin, urea).

As a carrier material or reservoir, in principle, any skin-friendly, physiologically acceptable and easily sterilisable materials can be used that can be impregnated with the above-mentioned substances or/and which are capable of absorbing these substances without any detrimental effect on the stability of the medicinal substance(s). Preference is given to absorptive, elastic materials. A certain absorbency is advantageous in respect of the absorption of water, of active substance-containing liquid (during manufacture) or of ichors.

Suitable carrier materials are, in particular, nonwovens, interlaced yarns or crocheted fabrics, knit fabrics, nonwovens, papers (e.g. filter papers, medicinal papers), absorbent gauze, wadding, and compresses, as well as combinations of the afore-mentioned materials, with cotton fabrics, viscose fabrics, cotton-viscose blended fabrics, synthetic fibre wovens, synthetic fibre nonwovens, cotton wadding and viscose wadding, pressed cotton and gauze-wadding compresses being especially preferred. Suitable as synthetic fibres are, in particular, polyesters, polyamides and polyurethane. The layer thickness of the carrier is preferably 0.1 to 10 mm, preferably 0.5 to 5 mm.

According to another embodiment, the inventive wound dressings or adhesive wound dressings contain at least one additive selected from the group comprising disinfectants, antioxidants, preservatives and moisture-absorbing substances. Substances suitable for this purpose are known to those skilled in the art. Suitable antioxidants are, in particular, tocopherols and the esters thereof, ascorbic acid, carotenes and carotenoids. Materials which can be used as substances with disinfectant effect are, for example, cetrimonium bromide, benzalkonium chloride, chlorhexidine, chlorhexidine derivatives and salts thereof.

Preferably, the inventive wound dressings are singly packed within an oxygen-impervious packaging material and are, in addition, protected against action of light. Suitable for this purpose are, in particular, composite packaging materials which are made up of, for example, polyethylene, aluminium or paper. Especially preferred is SURLYN® (Du Pont) as a packaging medium. Preferably, the wound dressings or adhesive wound dressings are vacuum-packed or packed under a protective atmosphere.

The present invention furthermore relates to an adhesive wound dressing for covering bleeding wounds which comprises an active substance-containing carrier material according to the above description, a backing layer connected thereto, and a detachable protective layer. The surface area of the backing layer is larger than that of the active substance-containing carrier, and at least that surface region of the backing layer which projects beyond the active substance-containing carrier is provided with an adhesive surface.

This, on the one hand, enables the adhesive attachment on the skin (that is, in the area of the skin surrounding the wound), and, on the other hand, the backing layer provides a protective cover towards the outside for the carrier impregnated with active substance and for the wound. The adhesive surface comprises an adhesive, especially pressure-sensitive adhesive, coating which covers the entire surface, or only part of the surface, of the backing layer.

Particularly advantageous is an embodiment where the mentioned adhesive surface of the backing layer projects beyond the active substance-containing carrier on all sides and forms an adhesive, especially a pressure-sensitive adhesive, margin.

The backing layer may be made from a rigid or flexible or elastic material. Materials suitable for the backing layer are metal foils or plastic films, or composite materials of two or more of the mentioned materials; metallised polymer films, preferably metallised with aluminium, being particularly preferred. Suitable plastic films are, above all, polyester films having a particularly high strength, such as polyethylene terephthalate and polybutylene terephthalate, and in addition other films of skin-compatible plastics, such as polyvinylchloride, ethylene-vinyl acetate copolymers, polyvinyl acetate, polyethylene, polypropylene, polyurethane and cellulose derivatives.

To produce the backing layer, it is also possible to use textile fabrics if, on account of the physical characteristics of the components of the carrier or reservoir impregnated with active substance, it need not be feared that the components penetrate through said textile material. Said textile fabric may be, for example, a woven fabric, interlaced yarns or crocheted fabric, a knitted fabric or a nonwoven consisting of the above-mentioned materials. Combinations or composite materials of textile fabrics and plastic films can also be utilised. The backing layer preferably has a layer thickness of from 0.01 mm to 2 mm, preferably 0.05 to 0.1 mm.

The adhesive surface or the adhesive margin is preferably formed by a pressure-sensitive adhesive layer consisting of a self-adhesive polymer matrix which may contain one or more additives. Apart from its pressure-sensitive adhesive property, the polymer matrix also has the property of ensuring or improving the cohesion of the adhesive wound dressing. Preferably, the pressure-sensitive adhesive layer has a self-tackiness that guarantees permanent contact with the skin.

The polymer matrix preferably contains a pressure-sensitive adhesive base polymer or a combination of at least two pressure-sensitive adhesive base polymers. Basically, any polymers are suitable for this purpose that are utilised in the production of pressure-sensitive adhesives, provided that they are physiologically acceptable and do not decompose adrenaline (or the medicinal substance(s) used in a given case).

In this connection, the polymer(s) is/are preferably selected from the group comprising natural rubbers, synthetic rubbers (e.g. rubber-like synthetic homopolymers, copolymers or block polymers), poly(meth)acrylic acid, poly(meth)acrylates, poly(meth)acrylate copolymers as well as combinations thereof. Especially preferred are styrene-diene copolymers, especially styrene-butadiene block copolymers, isoprene block polymers, acrylonitrile-butadiene rubbers, butyl rubbers or neoprene rubber, as well as silicone-based pressure-sensitive adhesives, and hot-melt adhesives.

The preferred acrylate-based polymers are, in particular, acrylate copolymers of 2-ethylhexyl acrylate-vinyl acetate and acrylic acid; preferred methacrylate-based polymers are, in particular, copolymers of dimethylaminoethyl methacrylate and neutral methacrylic acid esters.

The polymer matrix may preferably contain one or more additives; the selection of additives depends, inter alia, on the type of pressure-sensitive adhesive polymers used and on the active substance(s) used in each case.

These substances may, in particular, be additives from the group of the plasticisers, tackifiers, stabilisers, carrier substances and fillers. The physiologically acceptable substances suitable for this purpose are per se known to those skilled in the art. However, especially where adrenaline is used as the medicinal substance, one has to make sure that the polymers and additives which are used have a peroxide number that is as low as possible since peroxides are easily capable of decomposing the oxidation-sensitive adrenaline. Methods of determining the peroxide number are known to those skilled in the art.

Examples of suitable plasticisers are diesters of dicarboxylic acids as well as triglycerides, especially medium-chain triglycerides of caprylic acid/capric acid (e.g. of coconut oil); furthermore isopropyl myristate and isopropyl palmitate.

The total concentration of the additive(s) may be up to 70%-wt. and is preferably between 1 and 50%-wt., especially between 5 and 25%-wt., in each case relative to the adhesive matrix.

For the production of the detachable protective layer, which covers the pressure-sensitive adhesive layer and which is removed prior to use, the same materials are suitable as can be used for the production of the backing layer (see above), provided that the material has been rendered detachable, for example by silicone treatment. Other detachable protective layers are, for example, polytetrafluoroethylene, silicone-treated paper, cellophane, polyvinylchloride or similar materials.

It is particularly advantageous if the surface area of the protective layer is larger than the surface area of the individual adhesive wound dressing with which it is detachably connected. In particular, the protective layer may have a protruding end with the aid of which it can more easily be peeled from the adhesive wound dressing.

The inventive adhesive wound dressings are preferably packaged individually, in a packaging material that is impervious to oxygen and light (see above).

The present invention furthermore relates to processes for the production of the above-described wound dressings and adhesive wound dressings.

Such a process comprises at least the following steps:
Degassing a defined amount of a solvent or solvent mixture, or removing the oxygen therefrom, using a light-impermeable vessel.—Degassing may advantageously be effected by ultrasound treatment, or/and by gassing with nitrogen. As a result of the degassing, an oxidative destruction of medicinal substances, especially the adrenochrome reaction of the adrenaline, is suppressed. Another, or additional, measure consists in that for preparing the medicinal substance solution a solvent or solvent mixture is selected and provided which does not adversely affect the stability of a medicinal substance that is instable in the presence of oxygen. This is the case, for instance, if the solvent or solvent mixture is free of oxygen (or only contains traces thereof) and/or is free of peroxide, or has a very low peroxide content;
Adding a defined amount of at least one vasoconstrictive medicinal substance which is instable in the presence of oxygen or/and under action of light;
Dissolving the medicinal substance(s) in the solvent or solvent mixture;
Removing a partial amount of the solution and dripping the same onto the carrier material. In this manner, it is possible to meter a defined amount of the medicinal substance to the carrier material;
Drying and removing the solvent or solvent mixture; and
If required, repeating steps d) and e) in order to increase the proportion of the adsorbed medicinal substance.

During the entire process it should be taken into account to largely exclude an introduction of air or oxygen in the solution containing medicinal substance, and that the dripping and drying is carried out under exclusion of air, as much as possible, and preferably under protective gas. As protective gas, nitrogen may be used, for example; further suitable gases are known to those skilled in the art. When selecting the carrier material, one must likewise ensure that the peroxide content is low (if required, determination of the peroxide number; this number should preferably not exceed the value 10). If adrenaline (or salts thereof) is used as medicinal substance, the occurrence of oxidative decomposition during the production process can be easily detected because of the distinct red colouration of the decomposition product, which facilitates quality control during production.

The carrier material utilised in step (d), or the active substance-containing wound dressings made therefrom may, if necessary, be divided into formats appropriate for therapy, by cutting, punching or other known methods. Preferably, the inventive wound dressings and adhesive wound dressings are of an essentially circular, elliptical, square or rectangular shape, and the active substance-containing carrier preferably has a surface area of 1 to 100 cm², especially preferably 2 to 50 cm², more particularly 5 to 25 cm².

The carriers impregnated with active substance which are thus obtained can be singly packaged in the manner described above and are suitable for use as wound dressings.

An adhesive wound dressing may be produced by sticking an active substance-containing carrier, manufactured in accordance with the above-described process, to the adhesive surface of the backing layer, and subsequently covering the adhesive surface and the skin contact side of the active substance-containing carrier with a detachable protective layer.

According to another embodiment of the process, the adhesive wound dressings of the invention may be obtained by first applying a pressure-sensitive adhesive coating onto a surface of the particular material which is intended for the backing layer of the adhesive wound dressing. A piece of a carrier material is then placed on this adhesive surface of the backing layer. Subsequently, the active substance is metered—as described above—onto the carrier material and, after drying, the carrier material impregnated with active substance and the pressure-sensitive adhesive surface of the backing layer are covered with a detachable protective film.

The adhesive wound dressings according to the invention and the production thereof will be explained by the following examples of embodiments and the figures.

### BRIEF DESCRIPTION OF THE DRAWINGS

FIG. 1 is a schematic cross-sectional view of the adhesive wound dressing according to the invention, before addition of an adrenaline solution.

FIG. 2 is a plan view of the adhesive wound dressing according to the present invention.

FIG. 3 is a schematic cross-sectional view of the adhesive wound dressing of the present invention.

### DETAILED DESCRIPTION OF THE INVENTION

Production of an adrenaline-containing adhesive wound dressing:

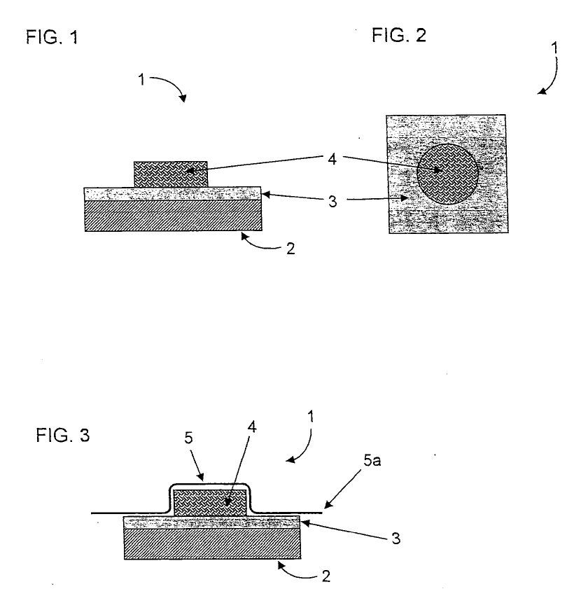

First, a pressure-sensitive adhesive layer is produced by coating a detachable paper with a pressure-sensitive adhesive solution. After removing the solvents by drying, the laminate, consisting of detachable paper and the dried pressure-sensitive adhesive layer, is covered with the layer which will later serve as the backing layer of the adhesive wound dressing. Then the detachable paper is removed and the pressure-sensitive adhesive layer is covered with a nonwoven, serving as a carrier material. The material used as the nonwoven is a nonwoven-fibre blend cotton/viscose staple fibre 70:30. Subsequently, a rectangular area of approximately 49 cm² is cut out of the pressure-sensitive adhesive layer lined with the nonwoven. Nonwovens having an adhesive matrix are depicted in FIGS. 1 and 2.

To prepare an adrenaline solution, 100 ml water for injection purposes is filled into a glass that is impermeable to light. The closed vessel is placed in an ultrasound bath for degassing. By ultrasound treatment for ten minutes, all the gases, especially air or aerial oxygen, are removed from the water.

25 g adrenaline acid tartrate is weighed into a second vessel, which is likewise impermeable to light, and is thereafter dissolved—while heating and stirring—in 75 ml of the degassed water.

Using a suitable device (e.g. dropper pipette) the adrenaline acid tartrate solution thus obtained is applied to the above-mentioned nonwoven. The moist nonwoven is dried for ten minutes under an infrared lamp, whereby the solvent (water for injection purposes) is completely removed. Immediately after the drying is stopped, the nonwoven and the pressure-sensitive adhesive layer are covered with a detachable protective layer. Then, the adhesive wound dressing (also called plaster) is punched to size, in such a manner that the backing layer and the pressure-sensitive adhesive layer, but not the protective layer, are punched through. The excess adhesive margin, consisting of backing layer and pressure-sensitive adhesive layer, is removed. The plaster thus obtained is immediately inserted into a three-side-sealed bag, and the open side thereof is immediately closed by welding. The finished product is depicted in FIG. 3.

FIG. 1 shows, in schematic cross-sectional view, the structure of an adhesive wound dressing (1) according to the invention, in the state before addition of the adrenaline solution. The adhesive wound dressing (1) is comprised of the backing layer (2), an adhesive layer (3) applied thereto, and a nonwoven (4) stuck to the adhesive layer.

FIG. 2 shows the adhesive wound dressing (1) depicted in FIG. 1, in plan view.

FIG. 3 shows, likewise in cross-section, the adhesive wound dressing (1) as the finished product. The nonwoven (4) is impregnated with adrenaline-containing solution (not shown). The nonwoven (4) and the pressure-sensitive adhesive layer (3) are covered with a detachable protective layer (5). The protective layer (5) projects beyond the surface of the backing layer, thus forming a protruding end as a gripping aid (5a). The sealed bag (package) is not shown.

The wound dressings and adhesive wound dressings according to the invention are advantageously suitable for treating bleeding wounds, especially for stopping the bleeding. They can be used both in the field of human medicine and in the field of veterinary medicine.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A wound dressing for covering bleeding wounds, said wound dressing being a ready-made product and comprising a carrier material containing a compound selected from the group consisting of adrenaline and one of the pharmaceutically acceptable salts of adrenaline, wherein said compound is a vasoconstrictive medicinal substance.

2. The wound dressing according to claim 1, wherein the carrier material contains at least one further vasoconstrictive medicinal substance selected from the group consisting of the sympathomimetics.

3. The wound dressing according to claim 1, wherein the carrier material further contains at least one substance selected from the group consisting of an astringent substance and a haemostatic substance.

4. The wound dressing according to claim 1, wherein the carrier material contains at least one further active substance for promoting wound healing but having no vasoconstrictive effect.

5. The wound dressing according to claim 4, wherein said at least one further active substance is selected from the group consisting of amino acids, peptides, enzymes, lymphokines, coagulation factors, anti-inflammatory substances, vitamins, polysaccharides and skin caring substances.

6. The wound dressing according to claim 1, wherein the carrier material is selected from the group consisting of wovens, interlaced yarns, crocheted fabrics, knit fabrics, nonwovens, papers, absorbent gauze, wadding, compresses, and combinations of said materials.

7. The wound dressing according to claim 6, wherein the carrier material has a low peroxide content.

8. The wound dressing according to claim 1 wherein the carrier material contains at least one additive selected from the group consisting of disinfectants, antioxidants, preservatives and moisture-absorbing substances.

9. An adhesive wound dressing for covering bleeding wounds, comprising an active substance-containing carrier material containing a compound selected from the group consisting of adrenaline and one of the pharmaceutically acceptable salts of adrenaline, a backing layer connected with the carrier material, and a detachable protective layer, the surface area of the backing layer being larger than the surface area of the carrier material, and wherein an adhesive surface comprises the surface area of the backing layer which projects beyond the surface area of the carrier material.

10. The adhesive wound dressing according to claim 9, wherein said adhesive surface of the backing layer projects beyond the carrier material on all sides to form an adhesive margin.

11. The adhesive wound dressing according to claim 9, wherein said backing layer comprises a material selected from the group consisting of a rigid material, a flexible material, an elastic material, and a composite material comprising at least two of said rigid flexible and elastic materials.

12. The adhesive wound dressing according to claim 9, wherein a pressure-sensitive adhesive layer comprising a polymer matrix having at least one additive forms one of said adhesive surface and said adhesive margin.

13. The adhesive wound dressing according to claim 12, wherein in that the polymer matrix contains one of a pressure-sensitive adhesive base polymer and a combination of at least two pressure-sensitive adhesive base polymers, the polymer(s) being selected from the group consisting of natural rubber, synthetic rubber, poly(meth)acrylic acid, poly(meth)acrylates, poly(meth)acrylate copolymers and combinations of said polymers.

14. The adhesive wound dressing according to claim 12, wherein the polymer matrix contains at least one additive selected from the group consisting of plasticisers, tackifiers, stabilisers, carrier substances and fillers.

15. The adhesive wound dressing according to claim 9, wherein said adhesive wound dressing is singly packed in an oxygen-impervious packaging material and is protected against the action of light.

16. A process for producing a wound dressing according to claim 1 or an adhesive wound dressing according to claim 9, the process comprising at least the steps of:
a) degassing a defined amount of a solvent or solvent mixture by using a light-impermeable vessel, or selecting and providing a solvent or solvent mixture which does not adversely affect the stability of a medicinal substance that is instable in the presence of oxygen;
b) adding a defined amount of a vasoconstrictive medicinal substance selected from the group consisting of adrenaline and one of the pharmaceutically acceptable salts of adrenaline;
c) dissolving the medicinal substance in the solvent or solvent mixture;
d) removing a partial amount of the solution and dripping the solution onto a carrier material;
e) drying and removing the solvent or solvent mixture; and
f) repeating steps d) and e), if required.

17. The process according to claim 16, further comprising the step of adding at least one further vasoconstrictive medicinal substance.

18. The process according to claim 16, further comprising the steps of:
g) sticking the carrier material containing the medicinal substance to an adhesive surface of a backing layer; and
h) covering the adhesive surface and the carrier material with a detachable protective layer.

19. A process for producing an adhesive wound dressing according to claim 9, further comprising the steps of:
i) coating a surface of a backing layer with a pressure-sensitive adhesive layer, or providing a backing layer having a pressure-sensitive adhesive surface;
ii) applying or sticking a carrier material to the adhesive surface of the backing layer;
iii) preparing an active substance-containing solution and dripping said solution onto a carrier material to form an active substance-impregnated carrier material; and
iv) covering the active substance-impregnated carrier material and the adhesive surface of the backing layer with a detachable protective film.

20. The process according to claim 19, further comprising the steps of:
punching out individual surface pieces having a defined surface shape and surface size; and
packaging the individual surface pieces in one package per piece, the package comprising an oxygen-impervious and a light-impermeable packaging material.

21. The process according to claim 19, wherein the dripping step is carried out under exclusion of air.

22. The process according to claim 19, wherein the carrier material has a low peroxide content.

23. Use of a wound dressing according to claim 1 or an adhesive wound dressing according to claim 19 for treating of bleeding wounds, by administering adrenaline to bleeding wounds to stop the bleeding.

24. Use of a vasoconstrictive medicinal substance for producing a ready-made wound dressing or an adhesive wound dressing for treating bleeding wounds, by using adrenaline or one of the pharmaceutically acceptable salts of adrenaline as the medicinal substance.

25. The wound dressing according to claim 5, wherein said amino acids are glycine.

26. The wound dressing according to claim 6, wherein the carrier material is selected from the group consisting of cotton fabrics, viscose fabrics, cotton-viscose blended fabrics, synthetic fibre wovens, synthetic fibre nonwovens, cotton wadding, viscose wadding, and gauze-wadding compresses.

27. The wound dressing according to claim 7, wherein the carrier material has a peroxide content not exceeding the value 10.

28. The adhesive wound dressing according to claim 11, wherein said material is selected from the group consisting of a metal foil and a plastic film.

29. The adhesive wound dressing according to claim 11, wherein said material is a metallised polymer film.

30. The adhesive wound dressing according to claim 29, wherein said metallised polymer film is a polymer film metallised with aluminium.

31. The wound dressing according to claim 1, wherein said wound dressing is singly packed in an oxygen-impervious packaging material and is protected against the action of light.

32. The process according to claim 21, wherein the dripping and drying is carried out under protective gas.

33. The process according to claim 16, wherein the dripping and drying steps are carried out under exclusion of air.

34. The process according to claim 33, wherein the dripping and drying steps are carried out under protective gas.

35. The process according to claim 22, wherein the carrier material has a peroxide number not exceeding the value 10.

36. The process according to claim 16, wherein the carrier material has a low peroxide content.

37. The process according to claim 36, wherein the carrier material has a peroxide number not exceeding the value 10.
